(19) United States
(12) Patent Application Publication
(10) Pub. No.: US 2024/0139135 A1
(43) Pub. Date: May 2, 2024

(54) PREGABALIN FOR TREATMENT OF OPIOID USE DISORDER

(71) Applicant: THE TRUSTEES OF INDIANA UNIVERSITY, Bloomington, IN (US)

(72) Inventor: Paul F. BUSTION, Anderson, IN (US)

(21) Appl. No.: 17/767,689

(22) PCT Filed: Oct. 9, 2020
(86) PCT No.: PCT/US2020/055011

§ 371 Date: Apr. 8, 2022

Related U.S. Application Data
(60) Provisional application No. 62/914,225, filed on Oct. 11, 2019.
(60) Provisional application No. 63/058,175, filed on Jul. 29, 2020.

(51) Int. Cl. A61K 31/197; A61K 31/542; A61K 31/635

(52) U.S. Cl. CPC A61K 31/197; A61K 31/542; A61K 31/635

(57) ABSTRACT

Disclosed herein is a method of treating opioid use disorder in a subject comprising administering an effective amount of pregabalin or a pharmaceutically acceptable salt thereof.

### CROSS REFERENCE TO RELATED APPLICATIONS

This application claims priority to the following: U.S. Provisional Application Ser. No. 62/914,225 filed on Oct. 11, 2019 and U.S. Provisional Patent Application Ser. No. 63/058,175 filed on Jul. 29, 2020, the disclosures of which are expressly incorporated by reference in their entirety.

### BACKGROUND

The present disclosure involves the discovery that pregabalin has efficacy with regard to treating opioid use disorder that was not previously known. Previously known uses of pregabalin include, for example, to manage fibromyalgia, diabetic nerve pain, or pain associated with diabetic peripheral neuropathy, spinal cord injury nerve pain, pain after shingles and, in combination with other seizure medicines, to treat partial onset seizures in patients with epilepsy. Reported advantages of pregabalin include that repeated use does not lead to tolerance, nor is there a cross-tolerance between morphine and pregabalin.

Disclosed herein are methods for terminating or decreasing the amount of opioid a subject is receiving and treating opioid use disorder (OUD).

Due to its increasing prevalence, OUD has recently has been declared a national health crisis in the U.S., though OUD is by no means limited to the U.S. This crisis has arisen in part as a result of the wide availability of opioids through the illegal narcotics trade, and in part as a result of legitimate medical uses of opioids. More particularly, OUD has arisen in many instances as a result of the prescription of opioids by physicians to treat patients for pain, whether short-term administration of an opioid to treat acute pain resulting from injuries or medical procedures, such as those occurring at surgical sites, or long-term administration of opioids to treat various forms of chronic pain. In all of these cases, use of opioids of the illegal or the pharmaceutical variety give rise to substantial risk of development of OUD, including opioid dependence and addiction. There is a need for new methods to reduce the risk of a patient developing OUD and new methods of treatment for patients diagnosed with OUD.

Treatment of OUD, which has at its aim the elimination of opioid dependence, opioid cravings and opioid addiction, is beset with substantial challenges. First, cessation of opioid use by a person having OUD is typically accompanied by serious, often excruciating, withdrawal symptoms. The avoidance of such withdrawal symptoms creates a strong disincentive to the cessation of opioid use, even among those who otherwise have a strong desire to cease opioid use. Second, opioids are highly addictive, creating a strong craving for opioids that very often is more powerful than a person's will power or desire to cease opioid use. Substantial resources have been devoted to efforts to overcome these challenges. Known treatment protocols for OUD are often associated with unfavorable side effects or outcomes, such as withdrawal symptoms, continued opioid use, or inadequate control of the pain for which the opioid was initially prescribed.

Prior efforts to develop treatment protocols for OUD have failed to appropriately eliminate the craving for opiates or to reduce such cravings to a level that enables successful cessation of opioid use in an acceptable percentage of cases. Thus, a need exists for OUD treatment protocols capable of eliminating or reducing the opioid cravings associated with OUD, preferably to a level that enables a person to discontinue opioid use, while simultaneously mitigating opioid withdrawal symptoms. With regard to those circumstances in which OUD has resulted from prescription and use of opiates to treat chronic pain, a need also exists for OUD treatment protocols capable of reducing the opioid cravings associated with OUD, preferably to a level that enables a person to discontinue opioid use, while simultaneously mitigating opioid withdrawal symptoms and controlling underlying pain. A need also exists for a post-surgical pain treatment protocol that includes the short-term use of opiates, which commonly are the most effective means of treating acute post-surgical pain, but such that patients can confidently utilize the most effective pain treatments without serious apprehension that they might develop OUD.

It also would be desirable to develop OUD treatment protocols that are based on one or more pharmaceutical compositions that have already gained regulatory approval (e.g., U.S. Food and Drug Administration (FDA) approval) and have been marketed. Examples of societal benefits of this approach would be reduced time and expense in obtaining regulatory approval for the new use of the pharmaceutical composition in OUD treatment protocols relative to a new composition and familiarity of the composition to prescribing physicians.

The present disclosure addresses these needs and provides other benefits and advantages.

### SUMMARY

The present disclosure provides a method of treating OUD in a subject comprising administering to a subject a therapeutically effective amount of a compound, or pharmaceutically acceptable salt thereof, of the formula

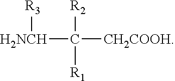

In some embodiments, the method further comprises synergistic use of a low dose of the compound in combination with a second compound. In some embodiments, the method further comprises administering a second compound of a non-opioid active agent to the patient. In some embodiments, the second compound is a non-steroidal anti-inflammatory drug (NSAID).

In some embodiments, the method further comprises synergistic use of a low dose of the compound in combination with an opioid receptor antagonist. In some embodiments, the method further comprises administering the low dose of the compound in combination with both the opioid receptor antagonist and an NSAID to the patient. In some embodiments, the opioid receptor antagonist comprises naloxone or naltrexone.

In some embodiments, the method comprises treating a patient who is displaying at least one symptom of OUD and receiving an opioid. In some embodiments, the method comprises treating a patient displaying a single symptom of OUD and receiving an opioid.

In some embodiments, a method of preventing a patient from developing OUD is provided. In some embodiments, the termination or reduction of opioid use by the patient prevents them from developing OUD. The present disclosure provides a method of terminating or decreasing the amount of opioid received by the patient comprising i) administering a compound, or pharmaceutically acceptable salt thereof, of the formula

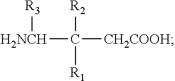

and ii) terminating or decreasing the amount of opioid received by the patient.

In some embodiments, the step of administering the compound occurs about two weeks after the patient began receiving the opioid.

In some embodiments, the methods reduce or eliminate opioid cravings in the patient. In some embodiments, the methods reduce or eliminate withdrawal symptoms in the patient.

In some embodiments, the methods provide administering a therapeutically effective amount of the compound. In some embodiments, the therapeutically effective amount is of a low dose. Such OUD treatment protocols advantageously are capable of eliminating or reducing the opioid cravings associated with OUD, leading to reduction or discontinuation of behaviors associated with opioid use, while also mitigating opioid withdrawal symptoms.

In some embodiments, the present disclosure provides a method of treating OUD or preventing the development of OUD in a patient comprising administering low doses of a compound of the formula

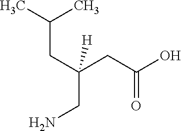

or a pharmaceutically acceptable complex, salt, solvate, or hydrate thereof.

In one aspect of the present disclosure, a method of treating opioid use disorder in a patient includes (i) diagnosing a patient with opioid use disorder and, (ii) administering a low dose of a compound to the patient over a treatment period; wherein the administering is effective to diminish, inhibit or eliminate behavior associated with cravings or use of said opioid. In another aspect, the disclosure provides a method of suppressing addictive effects of opioid use that includes (i) determining that a patient is in need of reducing or eliminating use of an opioid; and (ii) administering a low dose of the compound to the patient over a treatment period; wherein said administering is effective to suppress craving or addiction to opioids. In another aspect, the disclosure provides a method of preventing a patient from developing opioid use disorder including (i) administering the compound to the patient, wherein the patient is already receiving an opioid.

In some embodiments, the low dose comprises a daily dose of no greater than 75 mg of the compound. In one embodiment, a 25 milligram (mg) dose of the compound is administered thrice daily (T.I.D.) to a patient diagnosed with OUD. In some embodiments, the low dose comprises one or more daily doses. In some embodiments, the daily dose is administered as a single daily dose, as two smaller doses per day that total the daily dose, or as more than two smaller doses per day that total the daily dose. In some embodiments, the therapeutically effective amount is administered in at least two daily doses. In some embodiments, the low dose of the compound comprises a daily dose selected from no greater than 70 mg, no greater than 65 mg, no greater than 60 mg, no greater than 55, no greater than 50 mg, no greater than 45 mg, no greater than 40 mg, no greater than 35 mg, no greater than 30 mg, no greater than 25 mg, no greater than 20 mg, no greater than 15 mg, no greater than 10 mg, no greater than 5 mg, no greater than 4 mg, no greater than 3 mg, no greater than 2 mg, no greater than 1 mg, and no greater than 0.5 mg. In some embodiments, the treatment period is at least one day. In some embodiments, treatment period comprises a period of from one day to 30 days. In some embodiments, the compound is administered orally. In some embodiments, the compound is in a controlled-release form.

In some embodiments, the method also includes administering an NSAID in combination with the low dose of compound. In some embodiments, the method also includes administering one or more pain-control medication other than the compound in combination with the low dose of the compound. In some embodiments, the method includes orally administering the compound in combination with an NSAID, wherein the compound and the NSAID are combined in a single dosage form. In some embodiments, the dosage form comprises a capsule. In some embodiments, the single dosage form comprises no greater than an amount of the compound selected from the group consisting of 25 mg, 20 mg, 15 mg, 10 mg, 5 mg, 4 mg, 3 mg, 2 mg, 1 mg and 0.5 mg.

In some embodiments, the method also includes administering an opioid receptor antagonist in combination with the low dose of compound. In some embodiments, the method includes orally administering the compound in combination with the opioid receptor antagonist, wherein the compound and the one or more opioid receptor antagonists are combined in a single dosage form. In some embodiments, the dosage form comprises a capsule. In some embodiments, the single dosage form comprises no greater than an amount of the compound selected from the group consisting of 25 mg, 20 mg, 15 mg, 10 mg, 5 mg, 4 mg, 3 mg, 2 mg, 1 mg and 0.5 mg. In some embodiments, the one or more opioid receptor antagonist comprises naloxone or naltrexone. In some such embodiments, the method may further include administering an NSAID as described above in addition to the compound and the opioid receptor antagonist.

Additional embodiments, features, and characteristics of the present disclosure will be apparent from the following detailed description and through practice of the disclosure. The OUD treatment protocols of the present disclosure can be described as embodiments in any of the enumerated clauses set forth herein. It will be understood that any of the embodiments described herein can be used in connection with any other embodiments described herein to the extent that the embodiments do not contradict one another.

Clause 1. A method of treating a patient receiving an opioid and displaying at least one symptom of opioid use disorder comprising:
i. administering to the patient a therapeutically effective amount of a compound, or pharmaceutically acceptable salt thereof, of the formula

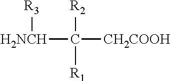

wherein R₁ is selected from a straight or branched alkyl of from 1 to 6 carbons, a phenyl, or a cycloalkyl having from 3 to 6 carbons; R₂ is hydrogen or methyl; and R₃ is selected from hydrogen, methyl, or carboxyl; and
ii. terminating or decreasing the amount of the opioid received by the patient.

Clause 2. The method of clause 1, wherein the step of administering the compound occurs at least about two weeks after the patient began receiving an opioid.

Clause 3. The method of clause 1, wherein the compound is a pharmaceutically acceptable salt of S-(+)-4-amino-3-(2-methylpropyl) butanoic acid, the salt being present as a single isomer.

Clause 4. The method of clause 1, wherein the compound, or pharmaceutically acceptable salt, is of the formula

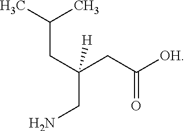

Clause 5. The method of clause 1 or 4, wherein the therapeutically effective amount is a low daily dose of no greater than about 75 mg.

Clause 6. The method of clause 1 or 4, wherein the therapeutically effective amount is administered as two daily doses.

Clause 7. The method of clause 1 or 4, wherein administering to the patient the therapeutically effective amount of the compound, or the pharmaceutically acceptable salt thereof, comprises orally administering to the patient the therapeutically effective amount of the compound, or the pharmaceutically acceptable salt thereof.

Clause 8. The method of clause 1 or 4, further comprising administering to the patient a second compound, wherein the second compound comprises a non-opioid active agent.

Clause 9. The method of clause 8, wherein the second compound is an NSAID.

Clause 10. The method of clause 9, wherein the NSAID is selected from Celebrex (celecoxib), Naprosyn (naproxen), or Mobic (meloxicam).

Clause 11. The method of clause 1 or 4, further comprising administering to the patient a second compound, wherein the second compound is an opioid antagonist.

Clause 12. The method of clause 11, wherein the second compound is selected from naloxone or naltrexone.

Clause 13. The method of clause 11, further comprising administering to the patient a third compound, wherein the third compound is an NSAID.

Clause 14. A method of treating opioid use disorder in a patient comprising:
i. administering to the patient a therapeutically effective amount of a compound, or pharmaceutically acceptable salt, of the formula

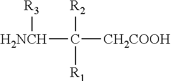

wherein R1 is selected from a straight or branched alkyl of from 1 to 6 carbons, a phenyl, or a cycloalkyl having from 3 to 6 carbons; R2 is hydrogen or methyl; and R3 is selected from hydrogen, methyl, or carboxyl.

Clause 15. The method of clause 14, wherein the step of administering occurs orally.

Clause 16. The method of clause 14, wherein treating opioid use disorder in the patient comprises treating a patient experiencing opioid cravings.

Clause 17. The method of clause 14, wherein the therapeutically effective amount is a low dose.

Clause 18. The method of clause 17, wherein the therapeutically effective amount is a single daily dose of about 0.5 mg/day to about 70 mg/day.

Clause 19. The method of clause 17, wherein the low dose is administered at least two times per day.

Clause 20. The method of clause 14, wherein the compound is of the formula

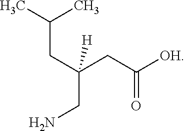

Clause 21. The method of clause 14 or 20, wherein the compound is administered orally.

Clause 22. The method of clause 14 or 20, further comprising administering a second compound.

Clause 23. The method of clause 22, wherein the second compound is an NSAID.

Clause 24. The method of clause 14 or 20, further comprising administering to the patient a second compound, wherein the second compound is an opioid antagonist.

Clause 25. The method of clause 24, wherein the second compound is selected from naloxone or naltrexone.

Clause 26. The method of clause 24, further comprising administering to the patient a third compound, wherein the third compound is an NSAID.

Clause 27. A method of treating a patient receiving an opioid and displaying at least one symptom of opioid use disorder, comprising:
i. administering to the patient a therapeutically effective amount of a compound of the formula

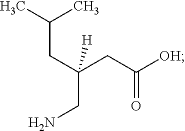

and
ii. terminating or decreasing the amount of the opioid received by the patient.

Clause 28. The method of clause 27, wherein the step of administering the compound occurs at least about two weeks after the patient began receiving an opioid.

Clause 29. The method of clause 27, wherein the therapeutically effective amount is a low daily dose of no greater than about 75 mg.

Clause 30. The method of clause 27, wherein the therapeutically effective amount is administered as three daily doses of about 25 mg.

Clause 31. The method of clause 27, wherein administering to the patient the therapeutically effective amount of the compound comprises orally administering to the patient the therapeutically effective amount of the compound.

Clause 32. The method of clause 27, wherein administering to the patient the therapeutically effective amount of the compound comprises administering to the patient the therapeutically effective amount of the compound in a controlled-release form.

Clause 33. The method of clause 27, further comprising administering to the patient a second compound, wherein the second compound comprises a non-opioid active agent.

Clause 34. The method of clause 33, wherein the second compound is an NSAID.

Clause 35. The method of clause 34, wherein the NSAID is selected from celecoxib or meloxicam.

Clause 36. The method of clause 34, wherein administering the NSAID in combination with therapeutically effective amount of the compound enhances the effects of the therapeutically effective amount of the compound.

Clause 37. The method of clause 27, further comprising administering to the patient a second compound, wherein the second compound is an opioid antagonist.

Clause 38. The method of clause 37, wherein the second compound is selected from naloxone or naltrexone.

Clause 39. The method of clause 37, further comprising administering to the patient a third compound, wherein the third compound is an NSAID.

Clause 40. A method of treating opioid use disorder in a patient comprising:
i. administering to the patient a therapeutically effective amount of a compound of the formula

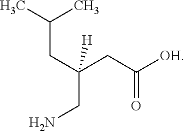

Clause 41. The method of clause 40, wherein administering to the patient the therapeutically effective amount of the compound comprises orally administering to the patient the therapeutically effective amount of the compound.

Clause 42. The method of clause 40, wherein treating opioid use disorder in the patient comprises treating a patient experiencing opioid cravings.

Clause 43. The method of clause 40, wherein administering to the patient the therapeutically effective amount of the compound comprises administering to the patient the therapeutically effective amount of the compound in a controlled-release form.

Clause 44. The method of clause 40, wherein the therapeutically effective amount is a low dose.

Clause 45. The method of clause 40, wherein the therapeutically effective amount is a single daily dose of about 75 mg.

Clause 46. The method of clause 40, wherein the therapeutically effective amount is a thrice-daily dose of about 25 mg.

Clause 47. The method of clause 40, further comprising administering a second compound.

Clause 48. The method of clause 47, wherein the second compound is an NSAID.

Clause 49. The method of clause 48, wherein administering the NSAID in combination with therapeutically effective amount of the compound enhances the effects of the therapeutically effective amount of the compound.

Clause 50. The method of clause 40, further comprising administering to the patient a second compound, wherein the second compound is an opioid antagonist.

Clause 51. The method of clause 50, wherein the second compound is selected from naloxone or naltrexone.

Clause 52. The method of clause 50, further comprising administering to the patient a third compound, wherein the third compound is an NSAID.

### DETAILED DESCRIPTION

For the purposes of promoting an understanding of the principles of the invention, reference will now be made to the embodiments described herein and specific language will be used to describe the same. The embodiments of the present application described below are not intended to be exhaustive or to limit the teachings of the present application to the precise forms disclosed in the following detailed description. It is also to be understood that the terminology used herein is for the purpose of describing particular embodiments only, and is not intended to be limiting. Rather, the terminology and embodiments are chosen and described so that others skilled in the art may appreciate and understand the principles and practices of the present application. It will therefore be understood that no limitation of the scope of the invention is intended by the description of specific embodiments. Any alterations and further modifications in the described embodiments, and any further applications of the principles of the invention as described herein are contemplated as would normally occur to one skilled in the art to which the invention relates.

Unless defined otherwise, all technical and scientific terms used herein have the same meaning as is commonly understood by one of ordinary skill in the art to which this disclosure belongs. Moreover, it should be understood that when certain values and ranges are recited herein in connection with various embodiments of the present teachings, all values and ranges which fall between such listed values and ranges are intended to be encompassed by the present teaching unless explicitly stated otherwise. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

As used herein, the terms “individual(s),” “subject(s),” and “patient(s)” mean any mammal. In some embodiments, the mammal is a human. In some embodiments, the mammal is a non-human.

The name of any given pharmacologically-active compound described herein should be understood to include corresponding chemical names and corresponding trade names by which the pharmacologically-active compound is marketed or otherwise known, alone or in combination with a pharmaceutically acceptable carrier to provide a pharmaceutical composition. A “pharmaceutically acceptable carrier” includes any of the standard pharmaceutical carriers, such as a phosphate buffered saline solution, water, emulsions such as an oil/water or water/oil emulsion, and various types of wetting agents. The term also includes solid carrier materials suitable for use in solid dosage forms. The term also encompasses any of the agents approved by a regulatory agency of the U.S. Federal Government or listed in the U.S. Pharmacopeia for use in animals, including humans.

As used herein, the term “pharmaceutically acceptable salt” refers to those salts, which counter ions which may be used in pharmaceuticals. Such salts include:
(1) acid addition salts, which can be obtained by reaction of the free base of the parent conjugate with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or
(2) salts formed when an acidic proton present in the parent conjugate either is replaced by a metal ion, e.g., an alkali metal ion, an alkaline earth ion, or an aluminum ion; or coordinates with an organic base such as ethanolamine, diethanolamine, triethanolamine, trimethamine, N-methylglucamine, and the like.

Pharmaceutically acceptable salts are well known to those skilled in the art, and any such pharmaceutically acceptable salt may be contemplated in connection with the embodiments described herein.

As used herein, the term “opioid use disorder” (“OUD”) includes opiate dependency syndrome, opioid addiction, opioid craving and similar conditions. The American Psychiatric Association provides four main categories with subcategories of symptoms to determine if a person has an opioid use disorder. If a person has at least two of these symptoms within a 12-month period of time, a doctor can diagnose someone as having an opioid use disorder. The symptoms are as follows: (1) Loss of Control; a) substance taken in larger amounts or for a longer time than intended, b) persistent desire or unsuccessful effort to cut down or control use of a substance, c) great deal of time spent obtaining, using, or recovering from substance use, and d) craving (a strong desire or urge) to use opioids; (2) Social Problems: a) continued opioid use that causes failures to fulfill major obligations at work, school, or home, b) continued opioid use despite causing recurrent social or personal problems, and c) important social, occupational, or recreational activities are reduced because of opioid use; (3) Risky Use: a) recurrent opioid use in dangerous situations and b) continued opioid use despite related physical or psychological problems; (4) Pharmacological Problems: a) tolerance (the need to take higher doses of a drug to feel the same effects or a reduced effect from the same amount) and b) withdrawal (the experience of pain or other uncomfortable symptoms in the absence of a drug).

A diagnosis of mild OUD requires the presence of 2-3 symptoms, moderate OUD requires 4-5 symptoms, and severe OUD requires 6 or more symptoms. However, the patient may have been diagnosed with OUD by other means, or may not have been diagnosed with OUD but nonetheless benefit from one or more of the OUD treatment protocols described herein.

As used herein, “opiate dependency syndrome” includes a need to continue taking the opiate to avoid withdrawal symptoms. The American Psychiatric Association defines withdrawal symptoms to include generalized pain, chills, cramps, diarrhea, dilated pupils, restlessness, anxiety, nausea, vomiting, insomnia, and intense cravings. Opiate dependency syndrome is a subset symptom of OUD. According to the American Psychiatric Association, symptoms of dependence can manifest after a person has taken an opioid for as little as two to four weeks.

As used herein, “opioid craving” or “cravings” includes a strong desire or strong urge to use opioids and is a subset symptom of OUD.

As used herein, the term “treating” includes administering an active agent for the purpose of eliminating a specific disorder, disease or condition; suppressing, diminishing, inhibiting, reducing, preventing or eliminating behaviors associated with a specific disorder, disease or condition; prophylaxis of a specific disorder, disease or condition; alleviating the symptoms associated with a specific disorder, disease or condition; and/or suppressing diminishing, inhibiting, reducing, preventing or eliminating said symptoms. For example, as used herein the term “treating OUD” will refer in general to administering an active agent for the purpose of eliminating OUD; suppressing, diminishing, inhibiting, reducing or eliminating behaviors associated with OUD; suppressing diminishing, inhibiting, reducing, or eliminating symptoms of OUD; suppressing diminishing, inhibiting, reducing, or eliminating a patient's dependence on opioid substances, addiction to opioid substances, craving for opioid substances or use of opioid substances and may include reducing or preventing withdrawal symptoms associated with the reduction or elimination of the patient's use of opioid substances.

As used herein, “treating OUD” or the like will generally include reducing or eliminating the use of opioid substances, dependence on opioid substances or craving for opioid substances by a patent who may not necessarily have OUD but for whom a reduction or elimination in opioid use otherwise is indicated. The term “treating OUD” may also encompass treatment protocols used with patients that are not using opioids but who may have a need for short-term opioid use and would benefit by use of methods described herein to reduce or eliminate apprehension associated with opioid use or reduce or eliminate the occurrence of opioid withdrawal symptoms by the patient following such short-term opioid use.

As used herein, “preventing the development of OUD” or the like will generally include preventing the development of dependence leading to withdrawal symptoms, cravings for opioids, risky use of opioids, or social problems related to opioid use.

As used herein, “low dose” refers to a dose of a pharmaceutical composition that is below the composition's therapeutic threshold for treating conditions other than OUD; e.g., below the composition's therapeutic threshold for treating neuropathic pain associated with diabetes, postherpetic neuralgia, or fibromyalgia. “Low dose” may be used interchangeably with “non-therapeutic dose.” Published evidence indicates that doses of pregabalin of up to 150 mg a day are consistently ineffective for the indicated treatment of neuropathic pain.

As used herein, “administration” generally means prescription or provision of a pharmaceutical composition to a patient for self-administration by the patient, and may also mean direct administration of a pharmaceutical composition to a patient by a clinician.

As used herein, the term “alkyl” includes a chain of carbon atoms, which is optionally branched and contains from 1 to 20 carbon atoms. It is to be further understood that in certain embodiments, alkyl may be advantageously of limited length, including C₁-C₁₂, C₁-C₁₀, C₁-C₉, C₁-C₈, C₁-C₇, C₁-C₆, and C₁-C₄. Illustrative alkyl groups include, but are not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like.

A s used herein, the term “cycloalkyl” refers to a 3 to 15 member all-carbon monocyclic ring, an all-carbon 5-member/6-member or 6-member/6-member fused bicyclic ring, or a multicyclic fused ring (a “fused” ring system means that each ring in the system shares an adjacent pair of carbon atoms with each other ring in the system) group where one or more of the rings may contain one or more double bonds but the cycloalkyl does not contain a completely conjugated pi-electron system. It will be understood that in certain embodiments, cycloalkyl may be advantageously of limited size such as C₃-C₁₃, C₃-C₆, C₄-C₆ and C₅-C₆. Cycloalkyl may be unsubstituted, or substituted.

As used herein, the term “aryl” refers to an all-carbon monocyclic or fused-ring polycyclic groups of 6 to 12 carbon atoms having a completely conjugated pi-electron system. It will be understood that in certain embodiments, aryl may be advantageously of limited size such as C₆-C₁₀ aryl. Illustrative aryl groups include, but are not limited to, phenyl, naphthalenyl and anthracenyl.

As used herein, the term “carboxyl” refers to a carbon atom that is double bonded to a and single bonded to a hydroxyl group.

As used herein, the term “hydroxyl group” or “hydroxy” refers to an —OH group.

It is appreciated that certain features of the disclosure, which, for clarity, may be described in the context of separate embodiments, may also be provided in combination in a single embodiment. Conversely, various features of the disclosure, which, for brevity, may be described in the context of a single embodiment, may also be provided separately or in any suitable subcombination.

The present disclosure is directed to OUD treatment protocols and other methods of treating OUD in a patient. In one embodiment, a method of treating OUD in a patient comprises administering to the patient a therapeutically effective amount of a compound, or pharmaceutically acceptable salt thereof, of the formula

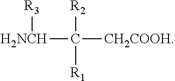

In some embodiments, a method of treating a patient receiving an opioid and displaying at least one symptom of opioid use disorder is provided. The method comprises
i. administering to the patient a therapeutically effective amount of a compound, or pharmaceutically acceptable salt thereof, of the formula

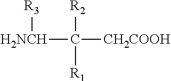

wherein R₁ is selected from a straight or branched alkyl of from 1 to 6 carbons, a phenyl, or a cycloalkyl having from 3 to 6 carbons; R₂ is hydrogen or methyl; and R₃ is selected from hydrogen, methyl, or carboxyl; and
ii. terminating or decreasing the amount of the opioid received by the patient.

In some embodiments, of the methods to treat a patient displaying at least one symptom of OUD or treating a patient for OUD, R₁ is selected from a straight or branched alkyl of from 1 to 6 carbons, a phenyl, or a cycloalkyl having from 3 to 6 carbons. In some embodiments, R₁ is a straight or branched alkyl of from 1 to 6 carbons. In some embodiments, R₁ is a phenyl. In some embodiments, R₁ is a cycloalkyl having from 3 to 6 carbons.

In some embodiments, R₂ is hydrogen or methyl. In some embodiments, R₂ is a hydrogen. In some embodiments, R₂ is a methyl.

In some embodiments, R₃ is selected from hydrogen, methyl, or carboxyl. In some embodiments, R₃ is a hydrogen. In some embodiments, R₃ is a methyl. In some embodiments, R₃ is carboxyl.

In some embodiments, the compound has the formula

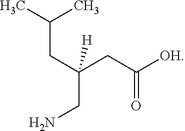

In some embodiments, the compound is S-(+)-4-amino-3-(2-methylpropyl) butanoic acid as a single isomer.

In some embodiments, the compound is pregabalin, also referred to as (S)-(+)-3-aminomethyl-5-methyl-hexanoic acid.

In some embodiments, the patient has opioid use disorder. In some embodiments, the patient has moderate to severe opioid use disorder. In some embodiments, the method to treat opioid dependence further comprises producing opioid abstinence.

In some embodiments, the methods to treat a patient displaying at least one symptom of OUD further comprises reducing opioid withdrawal symptoms. In some embodiments, the method to treat a patient displaying at least one OUD symptom further comprises reducing or eliminating opioid cravings.

One patient population for which such OUD treatment protocols and other methods are indicated includes opioid abusers who are to be withdrawn from opioids, such as those addicted to illegal opioid narcotics and patients within the moderate to severe range of scoring on the DSM-5 OUD criteria, suggesting that by clinician assessment such patients have a clinically significant issue with opioid use. Another patient population for which such OUD treatment protocols are indicated includes patients on opioids due to pain diagnosis and are to be withdrawn from opioids (e.g., due to patient request, intolerable side effects, or clinician assessment of opioid use disorder). The patients in this population may not necessarily have OUD but nonetheless may benefit from the OUD treatment protocols described herein. In another aspect, a patient population comprises patients for which it is desirable to prevent the development of OUD. In some embodiments, this patient population may be prescribed a dosing regimen of an opioid for at least about two weeks.

One aspect of the disclosure is a method of treating opioid use disorder in a patient or preventing the development of OUD in a patient that includes (i) diagnosing a patient with opioid use disorder or determining that a patient is in need of reducing or eliminating use of opioids, and (ii) administering a low dose of a compound to the patient over a treatment period. In some embodiments, the method further reduces or eliminate the patient's cravings for opioids.

In one aspect of the disclosure, the method comprises preventing the development of OUD in a patient comprising administering a therapeutically effective amount of a compound to the patient, wherein the patient has already received an opioid. In some embodiments, the patient is not displaying any symptoms of OUD. In some embodiments, the patient is displaying one symptom of OUD. In some embodiments the compound, or pharmaceutically acceptable salt thereof, is of the formula

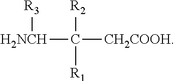

In some embodiments, R₁ is selected from a straight or branched alkyl of from 1 to 6 carbons, a phenyl, or a cycloalkyl having from 3 to 6 carbons. In some embodiments, R₁ is a straight or branched alkyl of from 1 to 6 carbons. In some embodiments, R₁ is a phenyl. In some embodiments, R₁ is a cycloalkyl having from 3 to 6 carbons.

In some embodiments, R₂ is hydrogen or methyl. In some embodiments, R₂ is a hydrogen. In some embodiments, R₂ is a methyl.

In some embodiments, R₃ is selected from hydrogen, methyl, or carboxyl. In some embodiments, R₃ is a hydrogen. In some embodiments, R₃ is a methyl. In some embodiments, R₃ is carboxyl.

In some embodiments, the compound has the formula

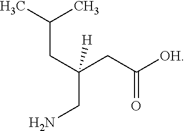

In some embodiments, the compound is S-(+)-4-amino-3-(2-methylpropyl) butanoic acid as a single isomer.

In some embodiments, the compound is pregabalin, also referred to as (S)-(+)-3-aminomethyl-5-methyl-hexanoic acid.

In some embodiments, the therapeutically effective amount is a low dose.

In some embodiments, the therapeutically effective amount of the compound to treat OUD or to prevent OUD in a patient is from about 0.5 mg/day to about 70 mg/day. In some embodiments, the low dose is a daily dose of no greater than 75 mg. In some embodiments, the low dose comprises a daily dose of no greater than an amount of compound selected from the group consisting of about 70 mg, about 65 mg, about 60 mg, about 55 mg, about 50 mg, about 45 mg, about 40 mg, about 35 mg, about 30 mg, about 25 mg, about 20 mg, about 15 mg, about 10 mg, about 5 mg, about 4 mg, about 3 mg, about 2 mg, about 1 mg, and about 0.5 mg.

In some embodiments, the daily dose is administered as a single daily dose, as two smaller doses per day that total the daily dose, or as more than two smaller doses per day that total the daily dose. In some embodiments, the therapeutically effective amount is administered in at least two daily doses. In some embodiments, an OUD treatment protocol includes administering low doses of the compound to a patient for the treatment of OUD at 25 mg T.I.D in replacement of opioids previously administered to the patient. The administration of the compound at 25 mg T.I.D. may be continued over a treatment period of one or more days as needed. In some embodiments, the treatment period comprises at least one day. In some embodiments, the treatment period is from about one day to about 60 days. In some embodiments, the treatment period comprises a period of from one day to 30 days.

In some embodiments, an OUD treatment protocol includes administering low doses of the compound in an oral dosage form to a patient. In some embodiments, an OUD treatment protocol includes administering low doses of a controlled-release or extended-release oral dosage form of the compound to a patient. Examples of suitable oral dosage forms contemplated by this disclosure include for example, and without limitation, those described in U.S. Pat. No. 10,022,447, which is hereby incorporated by reference herein in its entirety.

In some embodiments, the method further comprises administering the compound in a composition together with a pharmaceutically acceptable carrier.

In some embodiments, an OUD treatment protocol includes administering low doses of the compound in combination with NSAIDs or other non-opioid pain medications to a patient. In some embodiments, an OUD treatment protocol includes administering low doses of the compound to a patient in combination with an NSAID to enhance the effects of the compound and enable the use of lower doses of the compound in the OUD treatment protocol. In some embodiments, administering the low dose of the compound includes orally administering, the compound, such as, for example and without limitation, 25 mg the compound per dose, in combination with an NSAID, wherein the compound and the NSAID are combined in a single dosage form. In some embodiments, the dosage form comprises a capsule.

In some embodiments, an OUD treatment protocol includes administering 25 mg the compound T.I.D. to a patient in combination with an NSAID; e.g., Celebrex or Meloxicam, to enhance the efficacy of the low dose of compound, in which the compound and the NSAID are combined in correct proportions in a single capsule.

In some embodiments, an OUD treatment protocol includes administering low doses of the compound in combination with one or more opioid receptor antagonists. Opioid receptor antagonists are compounds that competitively bind to opioid receptors and have greater affinity to the opioid receptors than do opioids. Some opioid receptor antagonists; e.g., naloxone, block the effects of opioids and are commonly used to counteract depression of the respiratory and central nervous systems that occur during opioid overdose. Naloxone also may be combined with an opioid in preparations intended for oral administration to discourage individuals from abusing opioids by injecting the oral preparation. Due to low absorption when orally administered—but relatively high absorption when administered via injection—naloxone can block the effects of the opioid in such a preparation if it is injected while not interfering with the pain-relieving effects of the opioid when the preparation is administered orally as intended. Other opioid receptor antagonists; e.g., naltrexone, are used to block cravings for opioids.

In some embodiments, an OUD treatment protocol includes administering low doses of the compound to a patient in combination with an opioid receptor antagonist, such as (but not limited to) naloxone or naltrexone, to enhance the effects of the compound. In some embodiments, administering the low dose of the compound includes orally administering, the compound, such as, for example and without limitation, 25 mg the compound per dose, in combination with an opioid receptor antagonist, wherein the compound and the opioid receptor antagonist are combined in a single dosage form. In some embodiments, the dosage form comprises a capsule. In some embodiments, the OUD treatment protocol includes administering low doses of the compound to the patient in combination with the opioid receptor antagonists and an NSAID; e.g., Celebrex or Meloxicam.

In another aspect of the disclosure, there is provided a method of treating a patient displaying at least one symptom of OUD and a need for pain treatment that includes (i) determining that a patient is in need of treatment of an OUD symptom, wherein the patient displays the symptom of opioid craving and (ii) determining that the patient is in need of pain treatment; (iii) administering a low dose of a compound to the patient, wherein the compound, or pharmaceutically acceptable salt thereof, is of the formula

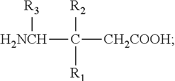

and
(iv) administering to the patient a non-opiate active agent in a dose that is effective to reduce pain; wherein administering the low dose of the compound to the patient reduces opioid craving and administering the non-opiate active agent to the patient reduces pain experienced by the patient. In some embodiments, the compound further mitigates opioid withdrawal symptoms.

In some embodiments, the low dose comprises a daily dose of no greater than 75 mg the compound. In some embodiments, the non-opiate active agent comprises an NSAID. In some embodiments, the NSAID comprises Celebrex (celecoxib), Naprosyn (naproxen), or Mobic (meloxicam). In some embodiments, the dose of the NSAID is a daily dose of from about 200 mg to about 400 mg of celecoxib, about 500 mg to about 1000 mg of naproxen or from about 7.5 mg to about 15 mg of meloxicam.

In some embodiments, R₁ is selected from a straight or branched alkyl of from 1 to 6 carbons, a phenyl, or a cycloalkyl having from 3 to 6 carbons. In some embodiments, R₁ is a straight or branched alkyl of from 1 to 6 carbons. In some embodiments, R₁ is a phenyl. In some embodiments, R₁ is a cycloalkyl having from 3 to 6 carbons.

In some embodiments, R₂ is hydrogen or methyl. In some embodiments, R₂ is a hydrogen. In some embodiments, R₂ is a methyl.

In some embodiments, R₃ is selected from hydrogen, methyl, or carboxyl. In some embodiments, R₃ is a hydrogen. In some embodiments, R₃ is a methyl. In some embodiments, R₃ is carboxyl.

In some embodiments, the compound has the formula

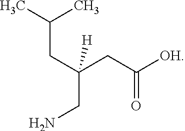

In some embodiments, the compound is S-(+)-4-amino-3-(2-methylpropyl) butanoic acid as a single isomer.

In some embodiments, the compound is pregabalin, also referred to as (S)-(+)-3-aminomethyl-5-methyl-hexanoic acid.

The discovery that a low dose of the compound functions to reduce or eliminate cravings of opioids further enable treatment protocols for treating acute pain using opioids with a significantly reduced risk of the use of opioids leading to a patient developing OUD. Another aspect of the present disclosure, is a method of treating a patient having a need for acute pain treatment that includes (i) administering an opiate to a patient in need of treatment for acute pain during a first period of time; and (ii) administering to the patient a low dose of a compound during a second period of time following the first period of time; wherein administering the low dose of the compound to the patient reduces or eliminates opioid craving and mitigates risks and symptoms associated with opioid use or withdrawal. In some embodiments, the acute pain is post-surgical pain, pain caused by a nonsurgical medical procedure, or pain caused by one or more non-medical injury. In some embodiments, the first period of time is a period of from about 2 days to about 2 months and wherein the second period of time is a period of from about 2 days to about 2 months.

In some embodiments, the compound comprises pregabalin. Pregabalin may be prepared using known methods. In some of these methods, a racemic mixture of 3-aminomethyl-5-methyl-hexanoic acid is synthesized and subsequently resolved into its R- and S-enantiomers. Such methods are described in U.S. Pat. No. 5,563,175 to R. B. Silverman et al., U.S. Pat. No. 6,046,353 to T. M. Grote et al., U.S. Pat. No. 5,840,956 to T. M. Grote et al., U.S. Pat. No. 5,637,767 to T. M. Grote et al., U.S. Pat. No. 5,629,447 to B. K. Huckabee & D. M. Sobieray, and U.S. Pat. No. 5,616,793 to B. K. Huckabee & D. M. Sobieray. In each of these methods, the racemate is reacted with a chiral acid (a resolving agent) to form a pair of diastereoisomeric salts, which are separated by known techniques, such as fractional crystallization and chromatography. In other methods, pregabalin is synthesized directly using a chiral auxiliary, (4R,5S)-4-methyl-5-phenyl-2-oxazolidinone. See, e.g., U.S. Pat. Nos. 6,359,169, 6,028,214, 5,847,151, 5,710,304, 5,684,189, 5,608,090, and 5,599,973, all to Silverman et al. In another method, pregabalin is prepared via asymmetric hydrogenation of a cyano-substituted olefin to produce a chiral cyano precursor of (S)-3-aminomethyl-5-methyl hexanoic acid, which is subsequently reduced to yield pregabalin. See U.S. Patent Application 2003/0212290 A1 to Burk et al.

The pharmaceutical composition wherein the compound is pregabalin may employ any pharmaceutically acceptable form of pregabalin, including its free form (zwitterion), and its pharmaceutically acceptable complexes, salts, solvates, hydrates, and polymorphs. In some embodiments, salts include, without limitation, acid addition salts and base addition salts, including hemisalts.

Pharmaceutically acceptable acid addition salts may include nontoxic salts derived from inorganic acids such as hydrochloric, nitric, phosphoric, sulfuric, hydrobromic, hydroiodic, hydrofluoric, phosphorous, and the like, as well as nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Potentially useful salts include acetate, aspartate, benzoate, chlorobenzoate, methylbenzoate, dinitrobenzoate, besylate, bicarbonate, carbonate, bisulfate, sulfate, pyrosulfate, bisulfite, sulfite, borate, camsylate, caprylate, citrate, edisylate, esylate, formate, fumarate, gluceptate, gluconate, glucuronate, hexafluorophosphate, hibenzate, hydrochloride, chloride, hydrobromide, bromide, hydroiodide, iodide, isethionate, isobutyrate, lactate, malate, maleate, malonate, mandelate, mesylate, methylsulfate, naphthylate, 2-napsylate, nicotinate, nitrate, orotate, oxalate, palmitate, pamoate, phosphate, hydrogen phosphate, dihydrogen phosphate, metaphosphate, pyrophosphate, phthalate, propionate, saccharate, sebacate, stearate, suberate, succinate, tartrate, tosylate, trifluoroacetate, and the like.

Pharmaceutically acceptable base salts may include nontoxic salts derived from bases, including metal cations, such as an alkali or alkaline earth metal cation, as well as amines Examples of potentially useful salts include, without limitation, aluminum, arginine, N,N′-dibenzylethylenediamine, calcium, chloroprocaine, choline, diethanolamine, diethylamine, dicyclohexylamine, ethylenediamine, glycine, lysine, magnesium, N-methylglucamine, olamine, potassium, procaine, sodium, tromethamine, zinc, and the like. For a discussion of useful acid and base addition salts, see S. M. Berge et al., J. of Pharm. Sci., 66:1-19 (1977); see also Stahl and Wermuth, Handbook of Pharmaceutical Salts: Properties, Selection, and Use (2002).

The pharmaceutically acceptable salts of pregabalin may be prepared by reacting its free (or zwitterionic) form with a desired acid or base; by removing an acid- or base-labile protecting group from a suitable precursor of pregabalin; by ring-opening a suitable cyclic (lactam) precursor using a desired acid or base; or by converting one salt of pregabalin to another by reaction with an appropriate acid or base or by contact with a suitable ion exchange column. All of these transformations are typically carried out in a solvent. The resulting salt may precipitate out and be collected by filtration or may be recovered by evaporation of the solvent. The degree of ionization in the resulting salt may vary from completely ionized to almost non-ionized.

Pregabalin may exist in unsolvated and solvated forms (including hydrates) and in the form of other multi-component complexes in which the drug and at least one additional component is present in stoichiometric or non-stoichiometric amounts. Multi-component complexes (other than salts and solvates) include clathrates (drug-host inclusion complexes) and pharmaceutical co-crystals. The latter are defined as crystalline complexes of neutral molecular constituents that are bound together through non-covalent interactions. Co-crystals may be prepared by melt crystallization, by recrystallization from solvents, or by physically grinding the components together. See, e.g., O. Almarsson & M. J. Zaworotko, Chem. Comm 1889-1896 (2004). For a general review of multi-component complexes, see J. K. Haleblian, J. Pharm. Sci. 64(8):1269-88 (1975).

Useful forms of pregabalin include all of its polymorphs and crystal habits, the corresponding R-enantiomer of pregabalin, and various mixtures of pregabalin and the R-enantiomer, including a racemic mixture of pregabalin and the R-enantiomer.

In addition, the pharmaceutical composition may employ prodrugs. In some embodiments, the pharmaceutical composition is pregabalin. Such prodrugs may be prepared by replacing appropriate functional groups of pregabalin with functionalities known as “pro-moieties,” as described, for example, in H. Bundgaar, Design of Prodrugs (1985). Examples of prodrugs would thus include derivatives of pregabalin in which an ester group replaces the carboxylic acid group or an amide group replaces the amino group.

This disclosure also contemplates that other compounds having structural similarity to pregabalin and similar activity as pregabalin (each such compound identified herein as a “related compound”) may be substituted for pregabalin in this disclosure. Examples of related compounds include, for example, other analogs of glutamic acid and gamma-aminobutyric acid (GABA) that are described in U.S. Pat. Nos. 6,001,876 and 6,197,819, each of which is incorporated herein by reference it its entirety. For purposes of this disclosure, it is understood that all aspects of the disclosure that reference pregabalin also constitutes disclosure of such aspect with each such related compound substituted for pregabalin.

### EXAMPLE

### Experimental Results

Summary of Experimental Results: Over the span of several years, the present inventor, who is a practicing neurologist, tested various different drugs at varying doses in an attempt to assist patients in his outpatient general neurological practice with cessation of opioid use. Through extensive experimentation, the physician discovered that 25 mg of Lyrica (pregabalin) T.I.D. successfully replaced opioids in a few of his patients, he subsequently expanded his research to other patients. After having successfully replaced opioids by way of this treatment for fifteen (15) patients who had been using opioids for at least three (3) months, with no reported side effects or withdrawal symptoms, the physician transmitted his records to an independent clinician for analysis. Each of these fifteen (15) patients was observed to exhibit a complete cessation of cravings for opioids.

While two (2) of the neurology patients in this fifteen-patient cohort elected to return to opioids for other reasons, these two patients also had been effectively cured of their cravings for opioids, and none of the remaining thirteen (13) patients who previously were addicted to opioids, and who had their opioids replaced with low doses of Lyrica (pregabalin), returned to opioids within the observation period. Additionally, none of the fifteen (15) patients experienced side effects or withdrawal symptoms. The experimental results indicate that the investigating physician has observed a relationship between the use of low doses of pregabalin and the ability to discontinue the use of opioids.

The charts for the fifteen (15) patients of this cohort were evaluated to ascertain the date that pregabalin was initiated, and the starting dose; the pregabalin dose as of the last available medical record; concomitant pain/CNS medications and concomitant non-pain/CNS medications; records of opioid use, type of opioid and dose (when available); and the temporal relationship between starting pregabalin and no longer requiring opioids. The medications in patient charts were compared with the accompanying medication list for each patient. As these were medical charts, the review was conducted to the extent that information was available. Data from this evaluation is summarized below.

Pain Diagnoses: All of these patients had painful conditions, with pain diagnoses that included chronic inflammatory demyelinating polyneuritis (1 patient), polyneuropathy (4 patients,) postoperative intercostal neuropathy (1 patient), migraines (4 patients), multiple sclerosis (2 patients), postherpetic neuralgia (1 patient), chemotherapy induced neuropathic pain (1 patient), cervical spondylosis (1 patient), diabetic neuropathy (2 patients), and chronic lumbar pain (1 patient). Several patients had overlapping diagnoses.

Diagnosis of opioid use disorder: As a part of the medical chart review, the investigating physician assessed the fifteen (15) patients against the DSM-5 diagnostic criteria for OUD, defined “ . . . as a problematic pattern of opioid use leading to clinically significant impairment or distress, as manifested by at least two (2) pre-defined symptoms.” These criteria include eleven symptoms or behaviors occurring within a 12-month period. A diagnosis of mild OUD requires the presence of 2-3 symptoms, moderate 4-5 symptoms, and severe 6 or more symptoms. Of the fifteen (15) patients, seven (7) had scores in the moderate range and eight (8) had scores in the severe range.

Timing of opioid use and discontinuation relative to the timing of starting pregabalin: Of the fifteen (15) patients, at least seven (7) stopped (five (5) patients) or decreased (two (2) patients) their opioids temporally related to (after) starting pregabalin. One (1) of these patients stopped oxycodone 10 mg q 6 h but continued taking tramadol 50 mg Q.I.D. prn. Of the remaining eight (8) patients, four (4) also stopped their opioids while on pregabalin, but the relative timing of stopping opioids and starting pregabalin could not be definitively established from the charts. Thus, of the patient charts reviewed, at least eleven (11) patients who were started on pregabalin stopped or decreased their use of opioids.

Doses of opioids: Opioid doses for this cohort of patients were either unspecified or were relatively low to moderate and included tramadol alone (two (2) patients), and oxycodone, oxycontin and dilaudid, alone or more typically in combination with other medications; e.g., NSAIDs, antidepressents, or antianxiolytics. There were also combinations of opioids, such as tramadol and oxycodone/apap. The patient taking oxycontin was on the highest opioid dose of 20 mg/30 mg every 8 hours with oxycodone/apap 10-325 mg every 6 hours.

Doses of pregabalin: Pregabalin is indicated for neuropathic pain (usual dose titrate to 300 mg/day, maximum 600 mg/day) associated with diabetes and postherpetic neuralgia (usual dose titrate to 300 mg/day, maximum 600 mg/day), and fibromyalgia (usual dose titrate to 300 mg/day, maximum dose 450 mg/day). (Pregabalin Package Insert, assessed June 2019). In the fifteen (15) patient cohort, at least nine were taking less than 300 mg per day.

Concomitant use of NSAIDs: Four (4) of the patients were confirmed to have been taking celecoxib (Celebrex) or diclofenac along with the pregabalin.

Chart Review Observations: The investigating physician's records from a population of patients with a variety of pain diagnoses, mainly neuropathic pain, display an observable relationship between use of pregabalin and the ability to discontinue the use of low-to-moderate-dose opioids.

Based upon the foregoing disclosure, it should now be apparent that the compound-based OUD treatment protocols described herein will carry out the objects set forth hereinabove. Namely, these compound-based OUD treatment protocols are capable of mitigating a patient's opioid withdrawal symptoms, reducing or discontinuing opioid use, and controlling pain. It is, therefore, to be understood that any variations evident fall within the scope of the present disclosure and thus, the selection of specific component elements can be determined without departing from the spirit of the invention herein disclosed and described.

We claim:

1. A method of treating a patient receiving an opioid and displaying at least one symptom of opioid use disorder, comprising:
i. administering to the patient a therapeutically effective amount of a compound of the formula 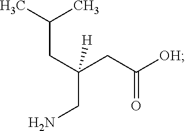
and
ii. terminating or decreasing the amount of the opioid received by the patient.

2. The method of claim 1, wherein the step of administering the compound occurs at least about two weeks after the patient began receiving an opioid.

3. The method of claim 1, wherein the therapeutically effective amount is a low daily dose of no greater than about 75 mg.

4. The method of claim 1, wherein the therapeutically effective amount is administered as three daily doses of about 25 mg.

5. The method of claim 1, wherein administering to the patient the therapeutically effective amount of the compound comprises orally administering to the patient the therapeutically effective amount of the compound.

6. The method of claim 1, wherein administering to the patient the therapeutically effective amount of the compound comprises administering to the patient the therapeutically effective amount of the compound in a controlled-release form.

7. The method of claim 1, further comprising administering to the patient a second compound, wherein the second compound comprises a non-opioid active agent.

8. The method of claim 7, wherein the second compound is a non-steroidal anti-inflammatory drug (NSAID).

9. The method of claim 8, wherein the NSAID is selected from celecoxib or meloxicam.

10. The method of claim 8, wherein administering the NSAID in combination with therapeutically effective amount of the compound enhances the effects of the therapeutically effective amount of the compound.

11. A method of treating opioid use disorder in a patient comprising:
i. administering to the patient a therapeutically effective amount of a compound of the formula

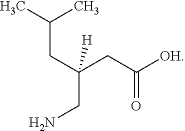

12. The method of claim 11, wherein administering to the patient the therapeutically effective amount of the compound comprises orally administering to the patient the therapeutically effective amount of the compound.

13. The method of claim 11, wherein treating opioid use disorder in the patient comprises treating a patient experiencing opioid cravings.

14. The method of claim 11, wherein administering to the patient the therapeutically effective amount of the compound comprises administering to the patient the therapeutically effective amount of the compound in a controlled-release form.

15. The method of claim 11, wherein the therapeutically effective amount is a low dose.

16. The method of claim 15, wherein the therapeutically effective amount is a single daily dose of about 75 mg.

17. The method of claim 15, wherein the therapeutically effective amount is a thrice-daily dose of about 25 mg.

18. The method of claim 11, further comprising administering a second compound.

19. The method of claim 18, wherein the second compound is an NSAID.

20. The method of claim 19, wherein administering the NSAID in combination with therapeutically effective amount of the compound enhances the effects of the therapeutically effective amount of the compound.
